(19) United States
(12) Patent Application Publication
(10) Pub. No.: US 2026/0309263 A1
(43) Pub. Date: Oct. 8, 2026

(54) PNEUMATIC ADAPTER

(71) Applicant: BELIMO HOLDING AG, Hinwil (CH)

(72) Inventors: Pascal GERNER, Wetzikon (CH); Frank LEHNERT, Rüti (CH); Patrick SCHMIDHEINY, Wetzikon (CH)

(73) Assignee: BELIMO HOLDING AG, Hinwil (CH)

(21) Appl. No.: 19/481,650

(22) PCT Filed: Jun. 6, 2024
(86) PCT No.: PCT/EP2024/065523

§ 371 Date: Nov. 5, 2025

(30) Foreign Application Priority Data
Jun. 9, 2023 (CH) CH000611/2023

(51) Int. Cl. F16L 39/02

(52) U.S. Cl. CPC F16L 39/02

(57) ABSTRACT

A pneumatic adapter for a Heating, Ventilation, and Air Condition (HVAC) sensor device includes an adapter body made of an elastomer, the adapter body having two inlet openings that extend into the adapter body in a main extension direction and that connect to two pneumatic ports of the HVAC sensor device, respectively, two outlet openings that extend into the adapter body and that connect to two pneumatic connectors of a sensor module of the HVAC sensor device that measures one or more characteristics of an HVAC fluid, and two channels that connect the two inlet openings to the two outlet openings, respectively.

### FIELD OF THE DISCLOSURE

The present disclosure relates to a pneumatic adapter for an HVAC sensor device for connecting two pneumatic ports of the HVAC sensor device to a sensor unit of the HVAC sensor device.

### BACKGROUND OF THE DISCLOSURE

HVAC (Heating, Ventilation, and Air Condition) sensor devices are used, as part of a HVAC system including pumps, chillers, heat pumps, piping, etc. to sense and thereby better control the climate of a facility. The HVAC sensor devices are in particular configured to measure characteristics of HVAC systems themselves and/or the facilities in which they are installed.

HVAC sensor devices are often connected to a fluid of an HVAC system, the fluid being, for example, a liquid or a gas. The fluid of the HVAC system may be air, for ventilation and/or climate control purposes. The fluid of the HVAC system may be water or another liquid for heating and/or cooling purposes, for example a mixture of water and antifreeze (e.g., glycol). The fluids are typically contained by and transported in ducts, channels, pipes, or tubes. The HVAC sensor devices may be installed adjacent to the fluid channel and connected to the fluid channel in order to measure characteristics of the fluid.

The HVAC sensor devices have sensor units designed for measuring one or more properties of the fluid. To this end, the HVAC sensor devices include an inlet for connecting the HVAC sensor device with the duct, channel, pipe, etc. Thereby, the fluid may enter the HVAC sensor device. The sensor units, which are often implemented as electronic devices attached to a circuit board of the HVAC sensor device, have a pneumatic connector and are connected to the inlet by way of flexible tubing, for example.

A disadvantage of using flexible tubing for connecting the sensor unit to the inlet is that the flexible tubing results in being a failure prone component of the HVAC sensor device. Additionally, it is cumbersome to fit during manufacture of the HVAC sensor device, typically requiring manual work.

### SUMMARY OF THE DISCLOSURE

It is an object of the disclosure and embodiments disclosed herein to provide a pneumatic interface assembly for connecting a pneumatic tube to a HVAC sensor device.

In particular, it is an object of the disclosure and embodiments disclosed herein to provide a pneumatic adapter for an HVAC sensor device, an HVAC sensor device including a pneumatic adapter, which does not have at least some of the disadvantages of the prior art.

The present disclosure relates to a pneumatic adapter for an HVAC sensor device. The pneumatic adapter comprises an adapter body made of an elastomer. The adapter body has two inlet openings extending into the adapter body in a main extension direction. The two inlet openings are configured to connect to two pneumatic ports of the HVAC sensor device, respectively. The adapter body has two outlet openings extending into the adapter body. The two outlet openings are configured to connect to two pneumatic connectors of a sensor unit of the HVAC sensor device. The sensor unit is configured for measuring at least one characteristic of an HVAC fluid. The adapter body has two channels connecting the two inlet openings to the two outlet openings, respectively.

In an embodiment, the adapter body is made of a form-stable elastomer.

In particular, the adapter body is made of Thermolast® K TC6MLZ, which is a thermoplastic elastomer. This material has a hardness of 59 on the Shore A hardness scale, meaning that it is a relatively soft material which has the benefit of improving a seal between the adapter body and the pneumatic connectors, and between the adapter body and the pneumatic sockets. Other materials with similar properties may also be employed.

In an embodiment, the adapter body is integrally formed, specifically in that it is made of, or formed into, a single piece. In other words, the adapter body is a single continuous component rather than two or more separate components, which may be connected to each other or joined together.

In an embodiment, in addition to its suitability for a HVAC sensor device, the pneumatic adapter may be suitable for use with an HVAC actuator, in particular a HVAC drive comprising an HVAC sensor device with an integrated actuator, for example an actuator comprising a motor for driving a valve.

In an embodiment, the adapter body comprises a plurality of pneumatic sockets. Two of the pneumatic sockets may define the two inlet openings extending into the adapter body in a main extension direction, respectively. Two of the pneumatic sockets may define the two outlet openings extending into the adapter body.

The adapter body may be composed entirely of the elastomer. The adapter body may, however, also be made in part of a second material different to the elastomer. The second material may be relatively more hard or form stable than the elastomer. The second material may be a plastic material, in particular an injection moulded polycarbonate such as Lexan™ FR Resin 916R.

In an embodiment, the adapter body comprises two pneumatic sockets defining the two inlet openings and made of the elastomer. The adapter body comprises two pneumatic sockets defining the two outlet openings and made of the elastomer. The adapter body comprises an interface member made of a material different than the elastomer, the interface member forming the two channels.

The plurality of pneumatic sockets may be attached to the interface member. The pneumatic sockets may be attached to the interface member by way of inserting the pneumatic sockets at least partially into the interface member.

Two of the pneumatic sockets define the two inlet openings extending into the adapter body in a main extension direction, respectively. Two of the pneumatic sockets define the two outlet openings extending into the adapter body.

The interface member may be made of a plastic material. The pneumatic sockets may be designed to receive a pneumatic plug. The pneumatic sockets may have an inner surface with a circular inner cross-section. The inner surface may have a varying diameter, for example a stepped inner diameter and/or a conically tapered inner surface.

At least two of the pneumatic sockets may be structurally interconnected such that they may be manufactured as a single piece, preferably using injection molding.

In an embodiment, the inlet openings are arranged on a first side of the adapter body. The outlet openings are arranged on a second side of the adapter body. The second side is arranged substantially perpendicular to the first side.

In an embodiment, the inlet openings are arranged on a first side of the adapter body. The outlet openings are arranged on a second side of the adapter body. The second side is arranged substantially parallel to the first side.

In an embodiment, the adapter body includes a bore extending through the adapter body in a direction perpendicular to the main extension direction of the inlet openings. The bore may be a partial bore or a through bore, i.e. extending partially through the adapter body or fully through the adapter body, respectively.

In an embodiment, the pneumatic adapter further comprises a rigid frame arranged on at least two lateral sides of the adapter body with respect to the main extension direction. The rigid frame is rigid in that it is more form-stable than the adapter body made of an elastomer. Specifically, the rigid frame is, at least in part, made of material which has a Young's modulus or hardness greater than that of the adapter body. The rigid frame is not necessarily entirely rigid but may be, or include parts which are, flexible or compressible In an embodiment, the rigid frame is made of plastic. The rigid frame may be injection-moulded plastic. For example, the rigid frame is made of Lexan™ FR Resin 916R, which is an injection moulded polycarbonate.

The rigid frame may be designed to increase the form-stability of the adapter body. The rigid frame may be designed to provide enhanced structural rigidity to the pneumatic adapter. The rigid frame may be designed such that the pneumatic adapter is graspable by an assembly robot. The plane may additionally serve to position and/or fix the pneumatic adapter to a circuit board, in particular a circuit board already having an installed sensor unit.

In an embodiment, the adapter body and the rigid frame are formed using two component molding or 3D printing. The adapter body and the rigid frame may be formed such that they are mechanically interlocking, i.e. cannot be pulled apart or separated from each other.

In an embodiment where the adapter body is made of two materials, in particular in which the adapter body comprises an interface member, the adapter body may form at least partially the rigid frame. Alternatively and/or additionally, at least some of the features described herein as being part of the rigid frame may be implemented or realized as part of the interface member.

In an embodiment, the adapter body has a stepped contacting surface. The stepped contacting surface may forms at least part of the second side. The second side may include the stepped contacting surface.

In an embodiment, the stepped contacting surface has a first section which includes the bore and a second section which includes the two outlet openings. The first section and the second section are preferably arranged plane parallel to each other. The first section may be designed for contacting a circuit board. The second section may be designed for abutting against at least part of the sensor unit.

In an embodiment, the two inlet openings extend, in the main extension direction, entirely through the adapter body forming two exit holes which are sealed by way of sealing plugs, respectively.

In addition to the pneumatic adapter described above, the present disclosure also relates to a pneumatic adapter for an HVAC sensor device. The pneumatic adapter comprises an adapter body made of an elastomer. The adapter body has two inlet openings which extend into the adapter body in a main extension direction. The two inlet openings are configured to connect to two pneumatic ports, respectively. The adapter body has two outlet openings which extend into the adapter body. The outlet openings are configured to connect to two pneumatic connectors of a sensor unit configured for measuring at least one characteristic of an HVAC fluid. The adapter body has a channel connecting a first inlet opening to a first outlet opening. The adapter body has three valve openings extending into the adapter body. The three valve openings are configured to connect to three pneumatic connectors of a three-way control valve. The first valve opening is connected to the second inlet opening. The second valve opening is connected to the second outlet opening. The third valve opening is connected to the channel.

The pneumatic adapter described above may include one or more of the features described with reference to the pneumatic adapter previously described.

In addition to the pneumatic adapters, the present disclosure also relates to an HVAC sensor device. The HVAC sensor devices comprises a sensor unit configured for measuring at least one characteristic of an HVAC fluid. The sensor unit is connected to a pneumatic adapter as described herein.

In addition to the pneumatic adapters, the present disclosure also relates to another HVAC sensor device. The HVAC sensor device comprises a sensor unit configured for measuring at least one characteristic of an HVAC fluid. The HVAC sensor device comprises a three-way control valve, wherein the sensor unit and the three-way control valve are connected to a pneumatic adapter as described herein.

The sensor unit may be configured to measure at least one of the following characteristics of an HVAC fluid: an absolute pressure, a differential pressure, a temperature, a CO2 concentration, volatile organic compounds (VOCs), or a particulate matter concentration.

### BRIEF DESCRIPTION OF THE DRAWINGS

The herein described disclosure will be more fully understood from the detailed description given herein below and the accompanying drawings, which should not be considered limiting to the disclosure described in the appended claims. The drawings in which:

FIG. 1 shows a exploded perspective view of components of an HVAC sensor device, including a pneumatic adapter, a rigid frame, a sensor unit, and a circuit board;

FIG. 2 shows a perspective view of FIG. 1 in which components of the HVAC sensor device, including the pneumatic adapter, are shown in an assembled state;

FIG. 3 shows the exploded perspective view of FIG. 1 from an alternative angle;

FIG. 4 shows a perspective view of FIG. 3 in which components of the HVAC sensor device, including the pneumatic adapter, are shown in an assembled state;

FIG. 5 shows a perspective view of a pneumatic adapter according to an embodiment;

FIG. 6 shows the perspective view of FIG. 5 with hidden lines and surfaces shown;

FIG. 7 shows three diagrams of an embodiment of a pneumatic adapter, including a side section view, a back view, and a top section view;

FIG. 8 shows a perspective view of an embodiment of a pneumatic adapter from the bottom front;

FIG. 9 shows a perspective view of the pneumatic adapter of FIG. 8 from the bottom back;

FIG. 10 shows an exploded perspective view of an HVAC sensor device including a pneumatic adapter, according to an embodiment;

FIG. 11 shows a partially exploded perspective view of the HVAC sensor device of FIG. 10;

FIG. 12 shows a perspective view of the HVAC sensor device of FIGS. 10 and 11;

FIG. 13 shows a perspective view of the HVAC sensor device of FIGS. 10 to 12, including two pneumatic adapters;

FIG. 14 shows a top section view of an HVAC sensor device according to an embodiment;

FIG. 15 shows a perspective view of a pneumatic adapter according to an embodiment;

FIG. 16 shows the perspective view of FIG. 15 from the bottom;

FIG. 17 shows diagrams of the pneumatic adapter of FIGS. 15, 16, including a bottom view, a front view, a bottom section view and a side section view;

FIG. 18 shows a variety of perspective views of a pneumatic adapter according to an embodiment; and

FIG. 19 shows diagrams of the pneumatic adapter of FIG. 18, including a bottom view and a front view.

### DESCRIPTION OF THE EMBODIMENTS

Reference will now be made in detail to certain embodiments, examples of which are illustrated in the accompanying drawings, in which some, but not all features are shown. Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

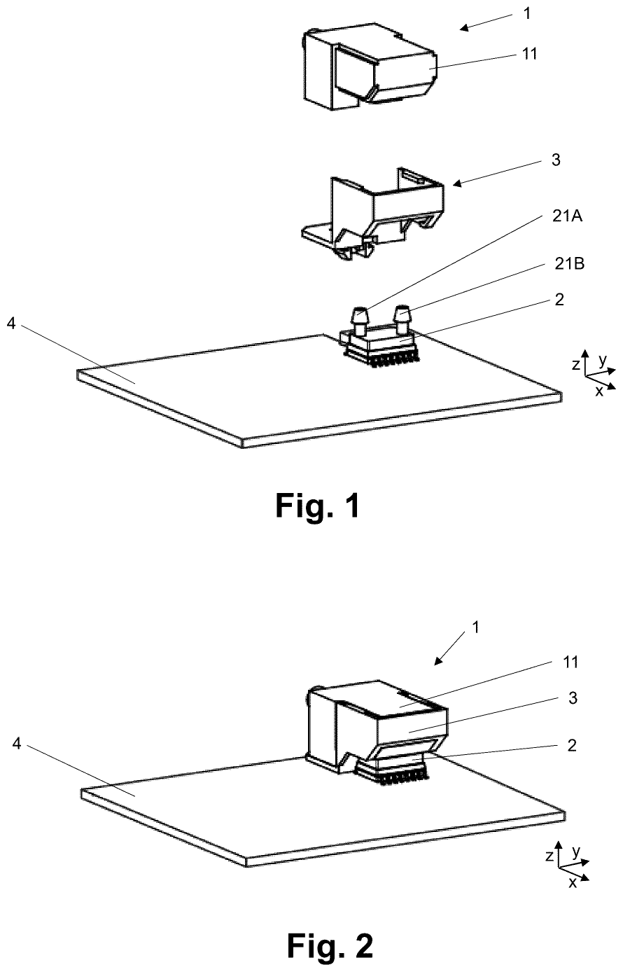

FIG. 1 shows an exploded perspective view of several components of an HVAC sensor device (not shown). A pneumatic adapter 1 is shown above a rigid frame 3. The rigid frame 3 is shown above a sensor unit 2. The sensor unit 2 is installed on a circuit board 4.

The pneumatic adapter 1 has an adapter body 11. The adapter body 11 is made of an elastomer, in particular a Underwriters Laboratories (UL) approved material. Preferably, the adapter body 11 has a Shore Hardness of ca. 40 to 60, more preferably 50, according to the Shore A scale. For example, the adapter body 11 is made of a silicon material, in particular a liquid silicone rubber (LSR), such as Momentive Silopren™ LSR 2050 or ELASTOSIL® LR 3003. In another example, the adapter body 11 is made of a thermoplastic elastomer (TPE).

The adapter body 11 is dimensionally stable such that it will return to its original shape after a force exerted on it ceases to be exerted on it. In other words, the adapter body 11, while being elastic due to being made of an elastomer, is not able to be significantly deformed, unlike a flexible hose, for example.

The adapter body 11 has two inlet openings and two outlet openings (not shown), which are, due to the dimensional stability, always located in substantially the same relative position to each other, unlike a flexible hose, for example, where the relative position of a hose inlet and hose outlet may be substantially altered upon application of a force.

The adapter body 11 may have a substantially cuboid shape. The substantially cuboid shape may include chamfers and/or bevels on one or more edges, and may include stepped surfaces.

The rigid frame 3 is designed to have a shape complementary to the shape of the pneumatic adapter 1, in particular to the shape of the adapter body 11. The rigid frame 3 is designed to at least partially enclose the pneumatic adapter 1. The rigid frame 3 is designed to establish an interference fit with the pneumatic adapter 1. For example, the rigid frame 3 has at least two opposing surfaces complementary to two opposing surfaces of the pneumatic adapter 1, such that the pneumatic adapter 1 fits snugly between the opposing surfaces. The rigid frame 3 is designed to have openings for the two inlet openings and the two outlet openings of the adapter body 11.

The rigid frame 3 may include fastening means for attaching the rigid frame 3 to the circuit board 4 and/or the sensor unit 2. In particular, the fastening means may include a snap fit mechanism designed to engage with an opening of the circuit board 4, such that when the rigid frame 3 is pressed onto the circuit board 4, the snap fit mechanism engages and securely attaches the rigid frame 3 to the circuit board 3.

The rigid frame 3 may be made of a plastic material, in particular an injection-moulded plastic material.

The sensor unit 2 is configured for measuring at least one characteristic of a fluid of an HVAC system. The characteristics include an absolute pressure, a differential pressure, a CO₂ concentration, a fine particular matter level, and/or a temperature. The sensor unit 2 includes transducers configured to generate an electric (i.e., analog and/or digital) signal output dependent on the measured characteristics of the fluid.

The sensor unit 2 includes two pneumatic connectors 21A, 21B which may be arranged on a top surface of the sensor unit 2. The sensor unit 2 is connected to the circuit board 4, for example via one or more soldered electrical connections.

During assembly of the HVAC sensor device, the sensor unit 2 is first attached to the circuit board 4, for example by soldering. The pneumatic adapter 1 may then be attached to the circuit board 4, either as one unit together with the rigid frame 3, or subsequently to the rigid frame 3 being attached to the circuit board 4. The pneumatic adapter 1 is attached to the circuit board 4 such that the outlet openings form a tight connection with the pneumatic connectors 21A, 21B.

FIG. 2 shows a perspective view of the components previously shown in FIG. 1, with the pneumatic adapter 1 fitted into the rigid frame 3. The rigid frame 3 is attached to the circuit board 4 on top of the sensor unit 2.

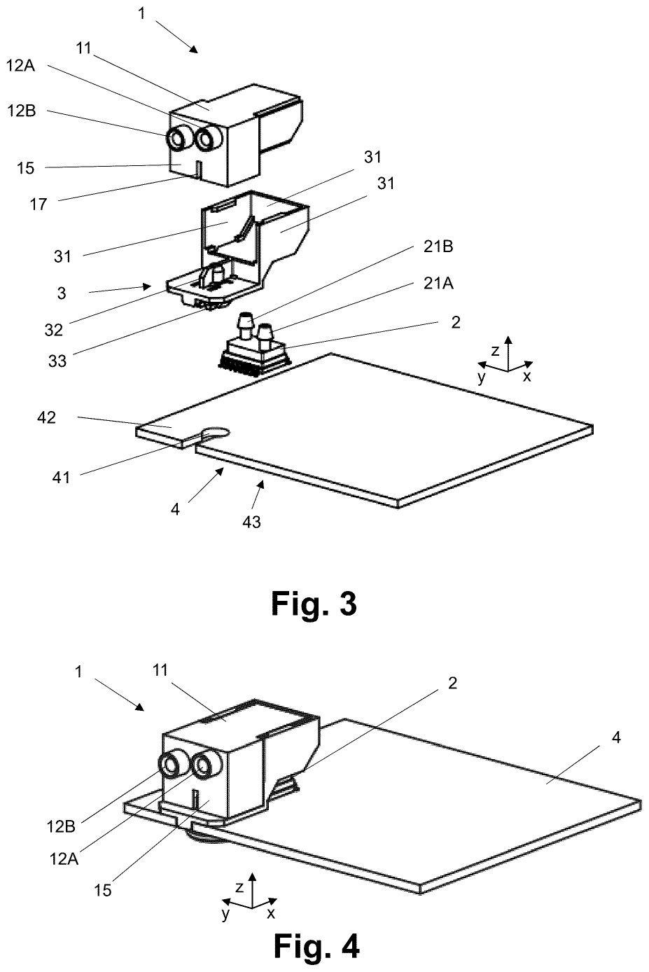

FIG. 3 shows an exploded perspective view of several components of an HVAC sensor device (not shown), similar to FIG. 1, however from a different angle. The adapter body 11 is shown including two inlet openings 12A, 12B on a first side 15 of the adapter body 11. The inlet openings 12A, 12B are circular cylindrical protrusions extending from a main volume of the adapter body 11. The inlet openings 12A, 12B are arranged adjacent to each other with a slight vertical displacement relative to each other.

The two inlet openings 12A, 12B extend, in the main extension direction x, into the adapter body 11, thereby forming channels (now shown).

The adapter body 11 includes, on a bottom surface, a recess 17. The recess 17 may include a bore (not shown). The recess 17 may be implemented as a slot.

The rigid frame 3 is shown having a plurality of surfaces 31 designed such that the adapter body 11 fits snugly onto and into the rigid frame 3.

The rigid frame 3 further includes engaging means 32 designed to engage with the adapter body 11 for a more secure fit. Preferably, the engaging means 32 are design to achieve a form fit with the recess 17 of the adapter body 11. The engaging means 32, may include a fin and/or a pin. The fin and/or pin are designed to engage with a slot and/or a bore of the adapter body, respectively.

The rigid frame 3 further includes, on a bottom surface, fastening means 33 for attaching the rigid frame 3 securely to the circuit board 4, in particular to a top surface 42 of the circuit board 4. The fastening means 33 include, for example, a snap fit mechanism. The snap fit mechanism is designed to securely attach the rigid frame 3 to the circuit board 4 by extending through an opening 41 of the circuit board 4 and engaging with a bottom surface of the circuit board 4.

The circuit board 4, for example implemented as a printed circuit board, has a top surface 42 and a bottom surface 43. An opening 41 in the circuit board 4 extends from the top surface 42 to the bottom surface 43. The opening 41 is designed such that the fastening means 33 of the rigid frame 3 can pass through the opening 41 when the rigid frame 3 is installed and hook themselves into the bottom surface 43, thereby securely attaching the rigid frame 3 to the circuit board.

FIG. 4 shows a perspective view of the components previously shown in FIG. 3, with the pneumatic adapter 1 fitted into the rigid frame 3. The rigid frame 3 is attached to the circuit board 4 on top of the sensor unit 2.

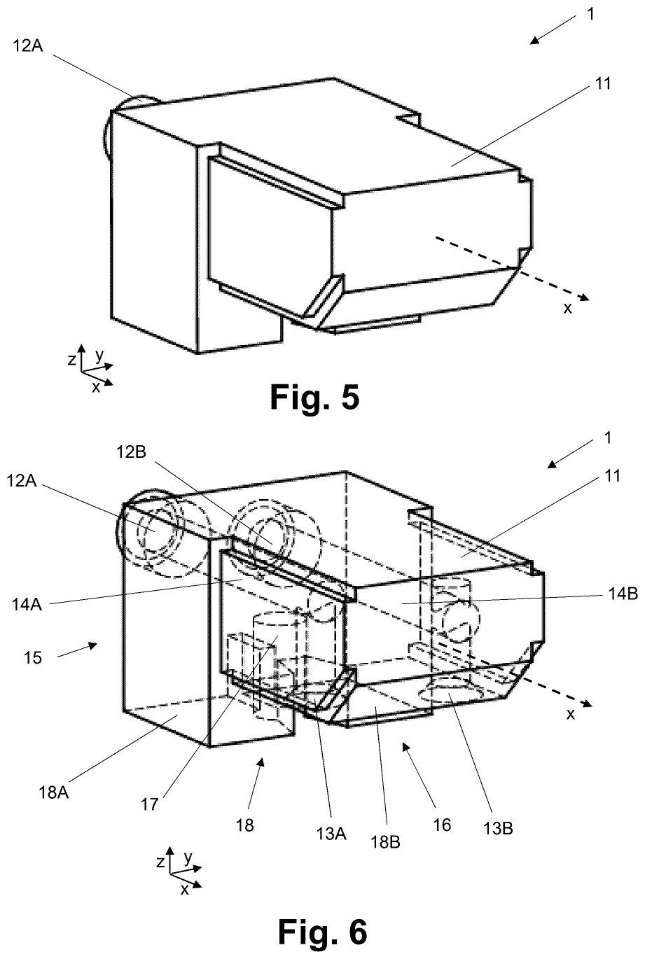

FIGS. 5 and 6 show close-up perspective views of the pneumatic adapter 1, with FIG. 6 showing hidden and interior lines as indicated by dashed lines. The inlet openings 12A, 12B are arranged on the first side 15 of the pneumatic adapter. The first side 15 is a substantially rectangular face of the pneumatic adapter 1. The inlet openings 12A, 12B extend into the pneumatic adapter 1 in the main extension direction x, thereby forming two channels 14A, 14B respectively.

In an embodiment, the channels 14A, 14B extend entirely through the adapter body 11, thereby forming two further openings. The two further openings may be closed off and/or otherwise sealed, for example by using sealing plugs.

Two outlet openings 13A, 13B are formed in the adapter body 11, on a second side 16 of the adapter body 1. The second side 16 is, in the embodiment shown, arranged perpendicular to the first side 15, however in other embodiments it may be arranged in parallel to the first side 16.

The outlet openings 13A, 13B extend into the adapter body. The two inlet openings 12A, 12B are connected to the two outlet openings 13A, 13B by way of the two channels 14A, 14B, such that a fluid, for example a gas or a liquid, can flow through the two channels 14A, 14B. The channels 14A, 14B may be bores or conduits. For example, the channels 14A, 14B may be implemented as circular cylindrical bores and, depending on the embodiment, may include one or more deviations, turns, and/or corners.

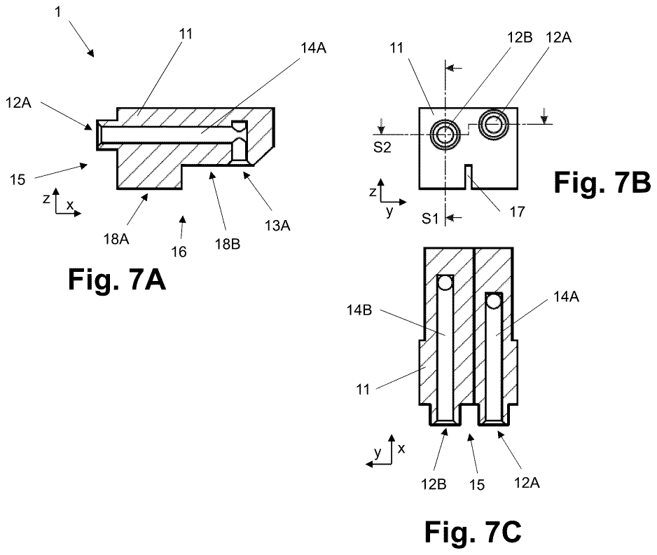

Alternatively, the two channels 14A, 14B may be designed to transmit pressure, such that a pressure at the two outlet openings 13A, 13B follows a pressure at the two inlet openings 12A, 12B, respectively. Specifically, the channels 14A, 14B are designed such that a pressure increase at the two inlet openings 12A results in a pressure increase at the outlet openings 13A, 13B, respectively. For example, the pressure at the outlet openings 13A, 13B may be proportional to the pressure at the inlet openings 12A, 12B, or even substantially the same. The channels 14A, 14B may be implemented as comprising channels 14A, 14B. The channels 14A, 14B may be semi-permeable to fluid, such that a flow of fluid through the channels 14A, 14B is at least partially restricted, slowed, and/or buffered. This may be implemented by including a foam-like material in the channels 14A, 14B. The channels 14A, 14B may be impermeable to fluid, such that the flow of fluid through the channels 14A, 14B is prevented while still allowing for at least a partial transmission of pressure through the channels 14A, 14B. This may be implemented by including a membrane inside the channel 14A, 14B, in particular a flexible membrane. FIGS. 7A, 7B, 7C show three section views of the pneumatic adapter 1, in particular the adapter body 11. FIG. 7B shows a section front view through the adapter body 11 in the y-z plane and the two channels 12A, 12B. Also shown in FIG. 7B are the cut lines S1, S2 for the section views shown in FIGS. 7A, 7C, respectively. The two channels 12A, 12B are offset from each other in the vertical z direction.

FIG. 7A, which is section side view in the x-z plane, shows the inlet opening 12A as formed as part of a protrusion on a first side 15 of the adapter body 11. The inlet opening 12A extends in the main direction x, thereby forming a channel 14A. The outlet opening 13A is on a second side 16 of the adapter body facing a negative z-direction. The outlet opening 13A is connected to the channel 14A.

The second side 16 includes two surfaces 18A, 18B which are offset from each other to form a stepped contacting surface.

FIG. 7C shows a section top view in the x-y plane showing the two channels 12A, 12B formed through the adapter body.

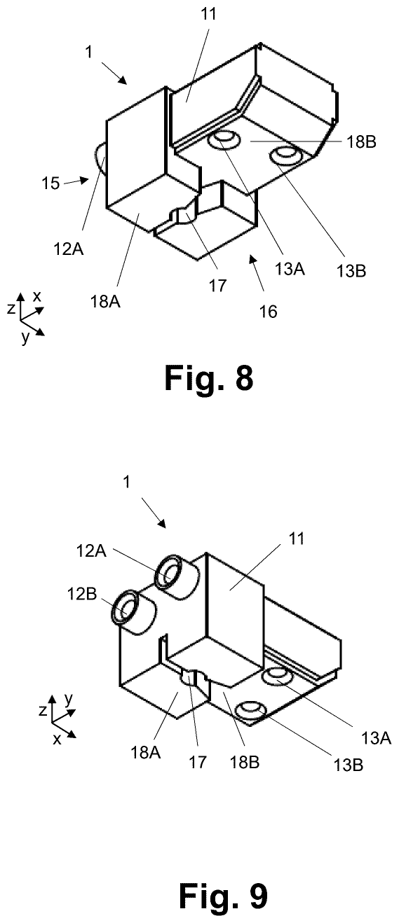

FIGS. 8 and 9 shown perspective views of the adapter body 11 of the pneumatic adapter 1, in particular from underneath the adapter body 11. The adapter body 11 is shown to include, on the second side 16 (in this case the bottom side), two surfaces 18A, 18B which are offset from each other in the vertical z direction, thereby forming a stepped contacting surface on which the adapter body 11 comes into contact with the circuit board 4 (not shown) when the pneumatic adapter 1 is installed. The two outlet openings 13A, 13B are on the surface 18B. The offset distance in the vertical z direction between the two surfaces 18A, 18B is designed to be substantially identical to a height of a main body of the sensor unit 2 (not shown), such that the surface 18A rests on the circuit board 4 and the surface 18B rests on the main body of the sensor unit 2, when the pneumatic adapter 1 is installed.

The recess 17, which may include a longitudinal slot extending in the main extension direction x and/or a bore in the z direction, is on the surface 18A.

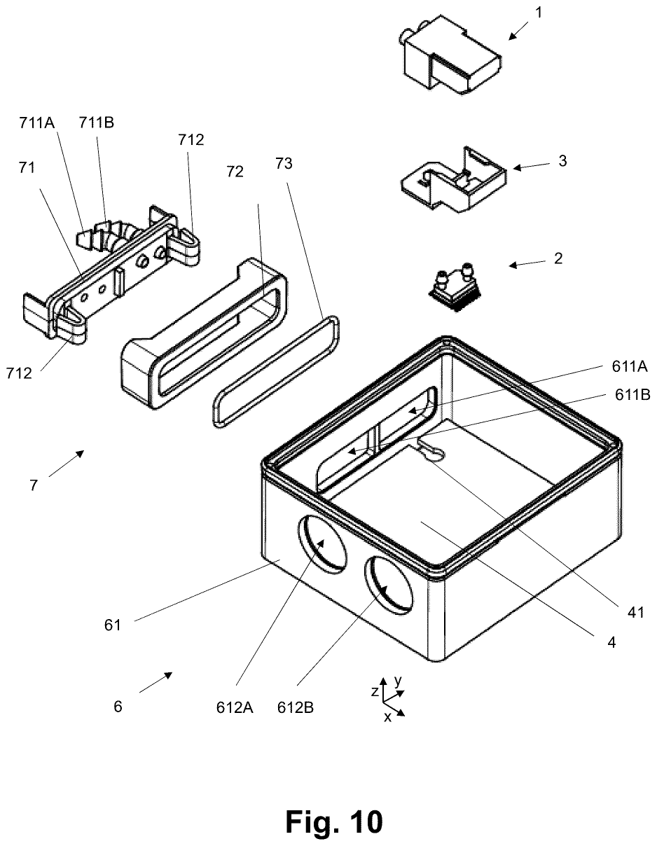

FIGS. 10 to 13 show various views of an HVAC sensor device 6 which includes the pneumatic adapter 1 as described herein.

FIG. 10 shows an exploded perspective view of an HVAC sensor device 6 including a pneumatic adapter 1. The HVAC sensor device 6 may be assembled as indicated in the Figure, i.e. in that particulars components, or groups of components, of the HVAC sensor device 6 are assembled as indicated by the sequence of particular components along a particular direction extending from the housing 61 of the HVAC sensor device 6.

For example, the circuit board 4 of the HVAC sensor device 6 is first installed in the housing 61. The sensor unit 2 is then installed onto the circuit board 4. The pneumatic adapter 1 is installed into the rigid frame 3, and then installed on the circuit board 4, on top of the sensor unit 2, together with the rigid frame 3.

In another example, an adapter 7 is installed. The adapter 7 includes an inlet housing seal 72, inlet shroud 72, and an inlet adapter plate 71. The inlet housing seal 73 is installed on an outside of the housing 61, such that it surrounds two housing inlet openings 611A, 611B. The inlet shroud 72 is then installed over the inlet housing seal 73. The inlet adapter plate 71 is then attached to the housing 61.

The housing 61 of the HVAC sensor device 6 has a substantially rectangular shape with a number of openings 611A, 611B, 612A, 612B for connections. The connections include connections to the fluid, however further optional connections, in particular electrical connections, may also be provided. In particular, electrical sockets may be fitted onto or through the openings 612A, 612B.

The inlet adapter plate 71 includes two pneumatic sockets 711A, 711B designed to connect, directly or indirectly, to a channel, duct, pipe, hose or other fluid carrying means of an HVAC system. The pneumatic sockets 711A, 711B include, on an inside face of the inlet adapter plate 71 (i.e. the side of the inlet adapter plate 71 facing an interior of the housing 61 when installed), protrusions designed to engage with the pneumatic adapter 1, in particular the inlet openings 12A, 12B of the adapter body 11, to achieve a tight seal.

In an embodiment, the two pneumatic sockets 711A, 711B form an acute angle with a surface normal of a plane of the inlet adapter plate 71, in particular such that they can be connected pneumatically from the same side as the electrical sockets which may be fitted on or through the openings 612A, 612B. For example, the two pneumatic sockets 711A, 711B point slightly in the negative y-direction as shown in FIG. 10. Thereby, the HVAC sensor device 6 can more easily be installed in tight spaces, as the pneumatic and electrical connections are attached from the same general direction.

The inlet adapter plate 71 further features engagement means 712 for securing the inlet adapter plate 71 to the housing 61. The engagement means 712 may be implemented as a snap fit mechanism, for example.

FIG. 11 shows a partially exploded perspective view of the HVAC sensor device 6. In particular FIG. 11 shows the pneumatic adapter 1 installed, together with the rigid frame 3, on top of the sensor unit 2, the sensor unit 2 being installed on the circuit board 4. The inlet adapter plate 71 is then installed, along with the inlet shroud 72 and the inlet housing seal 73, such that the pneumatic sockets 711A, 711B of inlet adapter plate 71 connect to the inlet openings 12A, 12B of the adapter body 11.

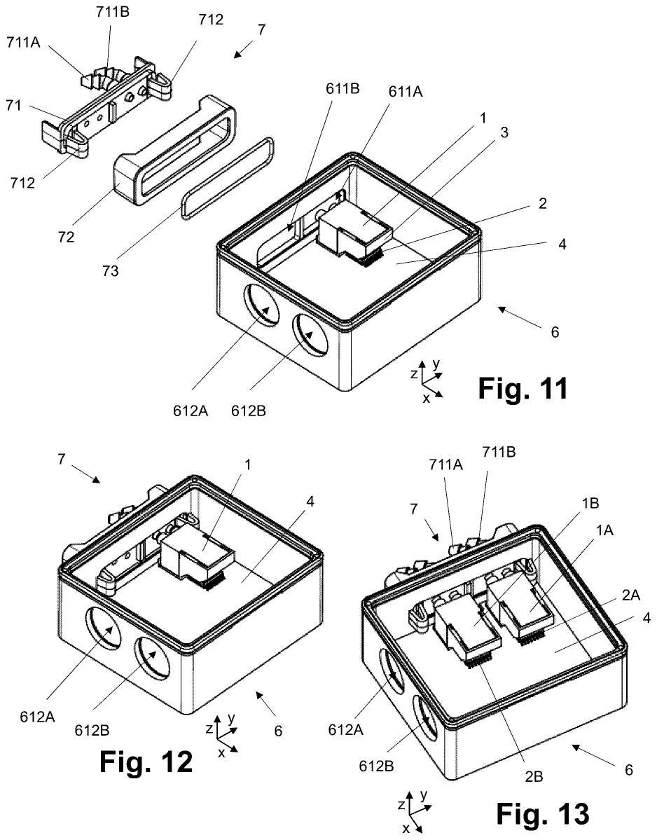

The HVAC sensor device 6 with assembled pneumatic adapter 1 and inlet adapter plate 71 is shown in FIG. 12. The assembly may be performed by robot, thereby significantly reducing the time needed to manufacture the HVAC sensor device 6.

FIG. 13 shows an embodiment in which the HVAC sensor device 6 includes two pneumatic adapters 1A, 1B, which are installed on top of two sensor units 2A, 2B, respectively. Accordingly, inlet adapter plate 71 also includes two sets of pneumatic sockets 711A, 711B.

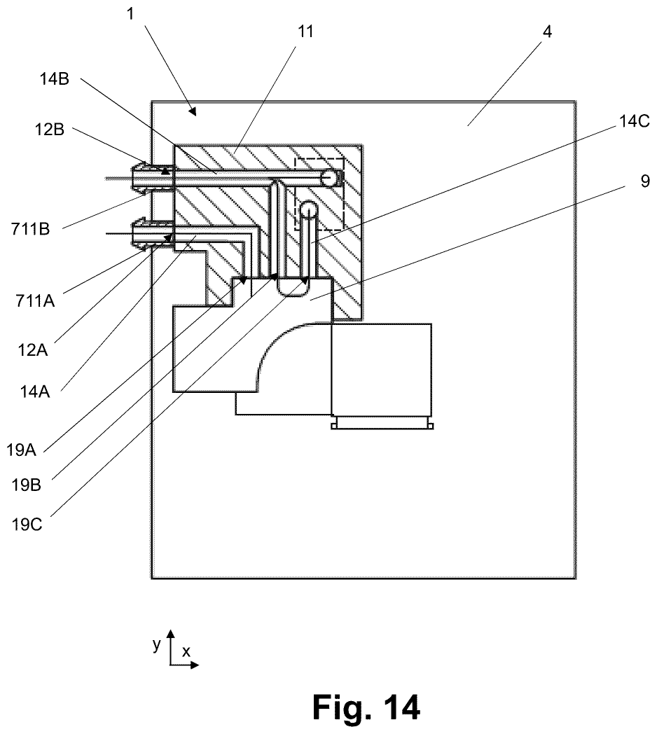

FIG. 14 shows a top section view of a pneumatic adapter 1 according an embodiment of the invention. The difference between the pneumatic adapter 1 shown in FIG. 14 with those shown in the other Figures is that the pneumatic adapter 1 is configured such that a valve 19 may be additionally connected to the pneumatic adapter 1.

For the sake of brevity, not all features (optional or not) of the pneumatic adapter 1 shown in FIG. 14 are explained. Rather, those features unique to the embodiment shown in FIG. 14 are explained. The person skilled in the art is aware that features from other embodiments in the present disclosure may be combined with the embodiment described with reference to FIG. 13.

The adapter body 11 includes a first valve opening 19A. The first valve opening 19A is connected to the first inlet opening 12A by way of a first channel 14A. The adapter body 11 includes a second valve opening 19B. The second valve opening 19B is connected to the second inlet opening 12B by way of a second channel 14B. The second channel 14B is further connected to the second outlet opening 13B (not shown). The adapter body 11 includes a third valve opening 19C. The third valve opening is connected to a third channel 14C which is connected to the first outlet opening 13A (not shown).

Thereby, the pneumatic adapter 1, in particular the adapter body 11, is configured such that a valve 19 may be opened to connect the two outlet openings 13A, 13B with each other via the adapter body 11 and the valve 19. This equalizes the pressure between the two outlet openings 13A, 13B. This may be performed in examples where the sensor unit 2 (not shown) includes a differential pressure unit and a zeroing is performed. The valve 19 may be controlled by the HVAC sensor device 6 to perform the zeroing.

The valve 19 may be designed to include three ports. A first valve port is designed to connect to the first valve opening 19A. A second valve port is designed to connect to the second valve opening 19B. A third valve port is designed to connect to the third valve opening 19C. In a closed state, the valve 19 connects the first valve port with the third valve port. The connection between the second valve port and the third valve port is closed. In an open state, the valve 19 connects the second valve port and the third valve port. The connection between the first valve port and the third valve port is closed.

The HVAC sensor device 6 may be configured to control the valve 19 to move between the closed position and the open position. In the closed position, the first inlet opening 12A is connected to the first outlet opening 13A (not shown) and the second inlet opening 12B is connected to the first outlet opening 13B (not shown). In the open position, the first inlet opening 12A is not connected to the first outlet opening 13A. Rather, in the open position, the second inlet opening 12B is connected to both the first outlet opening 13A and the second outlet opening 13B, thereby equalizing the pressure between the two outlet openings 13A, 13B and thereby achieving equal pressure at both sides of the sensor unit 2. In the closed position, the valve 19 provides for a normal functioning of the sensor unit 2, in particular a differential pressure unit which measures a pressure difference between the first inlet opening 12A and the second inlet opening 12B.

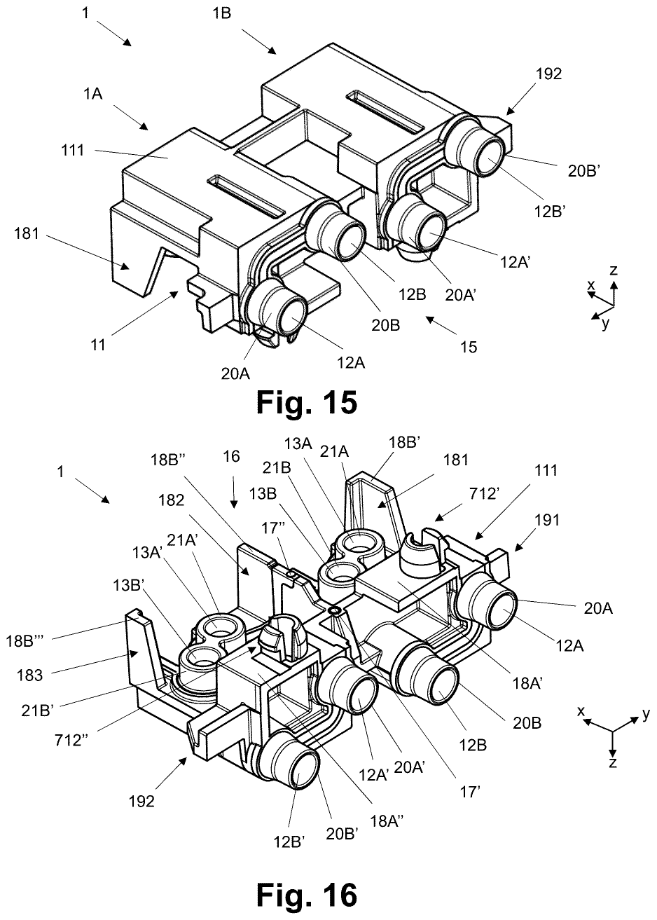

FIGS. 15 to 17 show various views of a pneumatic adapter 1 formed by using two-component molding. In particular, the pneumatic adapter 1 benefits from the two-component injection molding by using two materials in a molding of one part.

The adapter body 11 comprises a plurality of pneumatic sockets 20A, 20B, 20A′, 20B′, 21A, 21B, 21A′, 21B′ and an interface member 111 which implements at least some of the features of the rigid frame described herein. Throughout the description of these fig-ures, reference will be made to features described elsewhere as being part of the rigid frame. Within the context of these figures and the embodiments they describe, however, these features are part of the interface member 111. The pneumatic adapter 1 described with reference to FIGS. 15 to 17 is configured for two sensors.

The pneumatic sockets 20A, 20B, 20A′, 20B′, 21A, 21B, 21A′, 21B′ are made of the elastomer described herein. The pneumatic sockets 20A, 20B, 20A′, 20B′, 21A, 21B, 21A′, 21B′ have a substantially tubular shape and are attached to the interface member 111 by being partially inserted into the interface member 111.

FIG. 15 shows a top perspective view of the pneumatic adapter 1 configured for two sensors. FIG. 16 shows the pneumatic adapter 1 of FIG. 15 from a bottom perspective view.

The adapter body 11 comprises an elastomer, in particular a UL approved material. Preferably, the elastomer has a Shore Hardness of ca. 50 to 70, more preferably 59, according to the Shore A scale. For example, the elastomer is a thermoplastic elastomer, such as THERMOPLAST® K TC6MLZ.

The interface member 111 forms part of the adapter body 11 and is made of a plastic material. For example, the interface member 111 is made of an injection-molded plastic material, such as LEXAN™ FR Resin. As the relatively rigid interface member 111 forms a substantial portion of the adapter body 11,, the adapter body 111 maintains its stability. The interface member 111 provides enhanced structural rigidity to the adapter body 11 and can be graspable by an assembly robot. The design of the pneumatic adapter 1 thereby combines the advantages of increased stability of the adapter body 11 and improved airtightness of its pneumatic connections through the use of pneumatic sockets made of an elastomer in one part.

The pneumatic adapter 1 is configured for two sensors (not shown) and is implemented as two substantially identical sub-adapters 1A, 1B arranged side-by-side and connected to each other. Each sub-adapter 1A, 1B is designed for a single sensor. Therefore, the adapter body 11 implements, for a first of the two sensors, two inlet openings 12A, 12A′ on a first side 15 and two outlet openings 13A, 13A′ on a second side 16. For the second of the two sensors, the adapter body 11 includes two inlet openings 12B, 12B′ on the first side 15 and two outlet openings 13B, 13B′ on the second side 16. The inlet openings 12A, 12B, 12A′, 12B′ are implemented by four pneumatic sockets 20A, 20B, 20A′, 20B′, respectively. The outlet openings 13A, 13B, 13A′, 13B′ are implemented by four pneumatic sockets 21A, 21B, 21A′, 21B′, respectively.

The interface member 111 features, for each sub-adapter 1A, 1B, a bar defining a bar surface 18A′, 18A″. In particular, the bar stretches below (-z) the inlet openings 12A, 12A′, 12B, 12B′, without obstructing the outlet openings 13A, 13A′, 13B, 13B′. The interface member 111 includes two engagement means 712′, 712″, one for each sub-adapter 1A, 1B, which protrude from the bar surface 18A′, 18A″ of the interface member 111 for attaching the interface member 111 to a circuit board and/or a sensor. In particular, each engagement means 712′, 712″ may be implemented as a snap fit mechanism designed to engage with a respective opening of a circuit board, such that when the pneumatic adapter 1 is pressed onto the circuit board in the negative z-direction, the snap fit mechanisms engage and securely attach the pneumatic adapter 1 to the circuit board. While pressing the pneumatic adapter 1 onto the circuit board, it can be advantageous to connect two pneumatic connectors of the sensor to each outlet opening per sensor simultaneously (not shown).

The pneumatic adapter 1 also features a first, a second and a third leg 181, 182, 183 which have a substantially flat, fin-like profile and protrude from the interface member 111 in the vicinity of the outlet openings 13A, 13A′, 13B, 13B′. without enclosing the outlet openings 13A, 13A′, 13B, 13B′. The legs 181, 182, 183 contribute to the structural stability of the pneumatic adapter 1 and may also improve the security of the fit when the pneumatic adapter 1 is installed. The first 181 and third leg 183 are located at the opposite ends of the interface member 111 along the y-axis. The second leg 182 is located between the first outlet openings 13A, 13A′ and the second outlet openings 13B, 13B′ or between each sub-adapter 1A, 1B, defining a connection of sub-adapter 1A to sub-adapter 1B. Each leg 181, 182, 183 defines a leg surface 18B′, 18B″, 18B″ parallel to the second side 16 which extend to the same height as the bar surfaces 18A′, 18A″. Once the engagement means are connected to the circuit board, the bar surfaces 18A′, 18A″ and the leg surfaces 18B′, 18B″, 18B″ at least partially come into contact with the circuit board (not shown).

The pneumatic adapter 1 includes a first and a second recess 17′, 17″ which include a bore. Both recesses may be implemented as a slot. In particular, the recesses are located one at the leg surface 18B″ and one between the bars. The second recess 17′ may be located at a lower height in the negative z-direction than the first recess 17″.

The pneumatic adapter 1 may have a substantially cuboid shape or define an outer enveloping shape with is substantially cuboid. The substantially cuboid shape may include chamfers and/or bevels on one or more edged, and may include stepped surfaces. The pneumatic adapter 1 may also include grips 191, 192. For example, an assembly robot may grab and hold the pneumatic adapter 1 by the grips 191, 192. In particular, the grips form part of the interface member 111. The grips 191, 192 extend in the y-direction on opposing sides of the adapter body 1.

The pneumatic adapter 1 is configured for two sensors as mentioned above and includes two sub-adapters 1A, 1B, each designed for a single differential pressure sensor. The structural connection between the two sub adapters 1A, 1B to form the integral pneumatic adapter 1 is provided by the interface member 111, which extends from one sub-adapter to another. Further, the adapter body 11 may also be integrally formed in that the elastomer extends between both sub-adapters 1A, 1B. Consequently, at least one part of one sub-interface member of a sub-adapter may be combined with a substantially identical part of the sub-interface member 111.

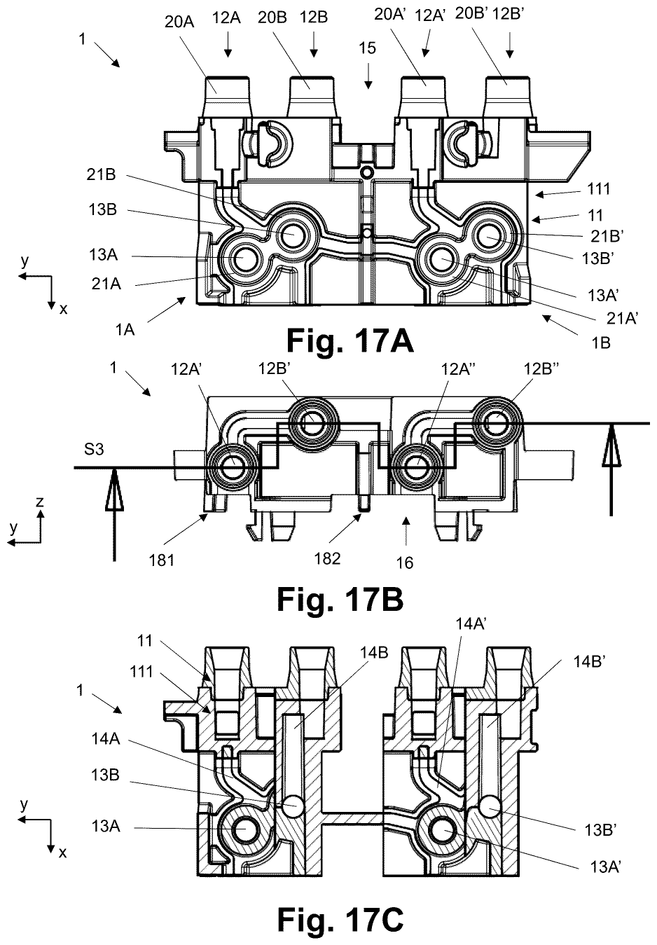

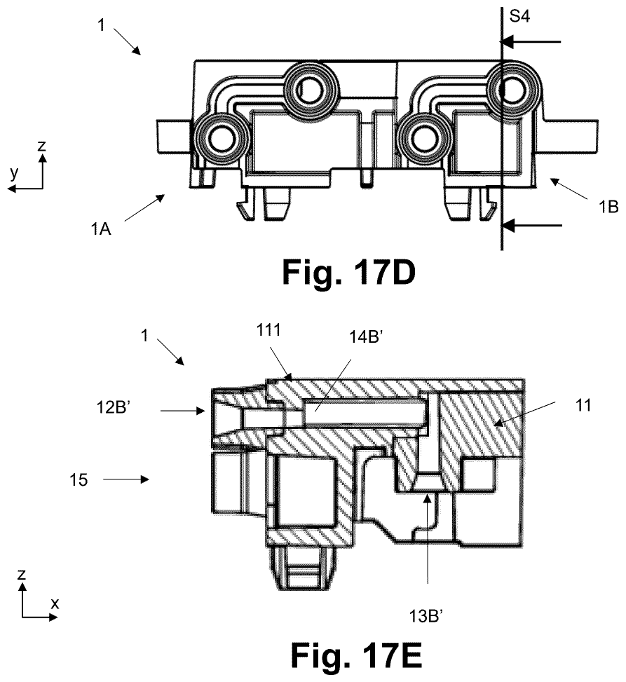

FIGS. 17A, 17B, 17C, 17D, 17E show the pneumatic adapter 1 formed by using two-component molding. It is to be noted that FIGS. 17B and 17D are the same figure, one indicating a cut line S3 and the other indicating a cut line S4. In the section views, the denser hatching indicates the adapter body 11 while the other hatching indicates the interface member 111.

FIGS. 17A, 17B show the pneumatic adapter 1 on the second side 16 and on the first side 15. In particular, the shape of the adapter body 11 is shown. At least some of the pneumatic sockets 20A, 20B, 20A′, 20B′, 21A, 21B, 21A′, 21B′ are connected to each other by one or more bridges. In particular, bridges may extend between the pneumatic sockets 20A and 20B, between the pneumatic sockets 20A′ and 20B′, between the pneumatic sockets 21A and 21B, and/or between the pneumatic sockets 21A′ and 21B′. The bridges are made of the same elastomer as the pneumatic sockets 20A, 20B, 20A′, 20B′, 21A, 21B, 21A′, 21B′ and improve the manufacturability of the adapter body, in particular for allowing the elastomer to flow easily through the mold during injection molding.

The bridges may feature straight and/or curved parts. In particular, the bridges on the second side 16, between inlet openings 12A, 12A′ and outlet openings 13A, 13A′ have for the most part in x-direction a straight shape and a curved shape in the vicinity of the outlet opening 13A, 13A′ and between outlet openings 13A, 13B and outlet openings 13A′, 13B′ have a substantially straight shape in y-direction. The bridges for each sub-adapter 1A, 1B on the first side 15, between inlet opening 12B, 12B′ and inlet openings 12A, 12A′ have for the most part in y-direction a straight shape and a curved shape in the vicinity of the inlet openings 12A, 12A′.

The outlet openings for each sub-adapter are arranged with an offset in x- and y-direction from each other and are configured to connect to two pneumatic connectors of a sensor unit configured for measuring at least one characteristic of an HVAC fluid, in particular a differential pressure across both outlet openings. Each inlet opening per sub adapter is arranged with an offset in z- and y-direction from others in order to facility ease of pneumatically connecting the inlet openings by increasing the geometric distance between the inlet openings.

FIG. 17C is a section side view in the x-y plane of the second side 16 and FIG. 17E is a section side view in the x-z plane. Both figures show the adapter body 11 as partially nested in the rigid frame 3. The inlet openings 12A, 12B, 12A′, 12B′ are formed as part of a protrusion of the adapter body 11. The pneumatic adapter 1 defines, in particular for each sub-adapter, a plurality of channels 14A, 14B′, 14A′, 14B′ which extend between the inlet openings 12A, 12B, 12A′, 12B′, and the outlet openings 13A, 13B, 13A′, 13B′, thereby fluidically connecting the inlet openings and outlet openings.

The walls of the channels 14A, 14B, 14A′, 14B′ may be defined by the adapter body 11, in particular by the pneumatic sockets 20A, 20B, 20A′, 20B′, 21A, 21B, 21A′, 21B′ and the interface member 111. In particular, the adapter body 11 may designed to define the channels 14A, 14B, 14A′, 14B′ only near and/or at the inlet openings 12A, 12B, 12A′, 12B′ and near and/or at the outlet openings 13A, 13B, 13A′, 13B′, while the interface member 111 defines at least part of a particular channel 14A, 14B, 14A′, 14B′ between the inlet openings 12A, 12B, 12A′, 12B′ and the outlet openings 13A, 13B, 13A′, 13B′. In other words, the interface member 111 includes bores which form part of the channels 14A, 14B, 14A′, 14B′. In effect, the pneumatic adapter 1 thereby provides inlet and outlet openings which are made of an elastomer and are therefore relatively soft, providing for a sufficiently good pneumatic seal (e.g., they are air-tight under normal operating conditions), while at least part of the channels in the interior of the pneumatic adapter 1 are formed of a relatively harder plastic (i.e., relatively harder than the elastomer of the adapter body 11). Thereby, the manufacturability and form stability of the pneumatic adapter 1 is improved.

For improving the pneumatic sealing capability of the inlet and outlet openings 12A, 12B, 13A, 13B, the channels are tapered, e.g. have a stepped and/or conical profile.

The overall diameter or interior cross section of the channels may vary, in particular the channels may have a larger cross section near the inlet and/or the outlet openings, while the channel may be narrower in the interior or bulk of the pneumatic adapter 1.

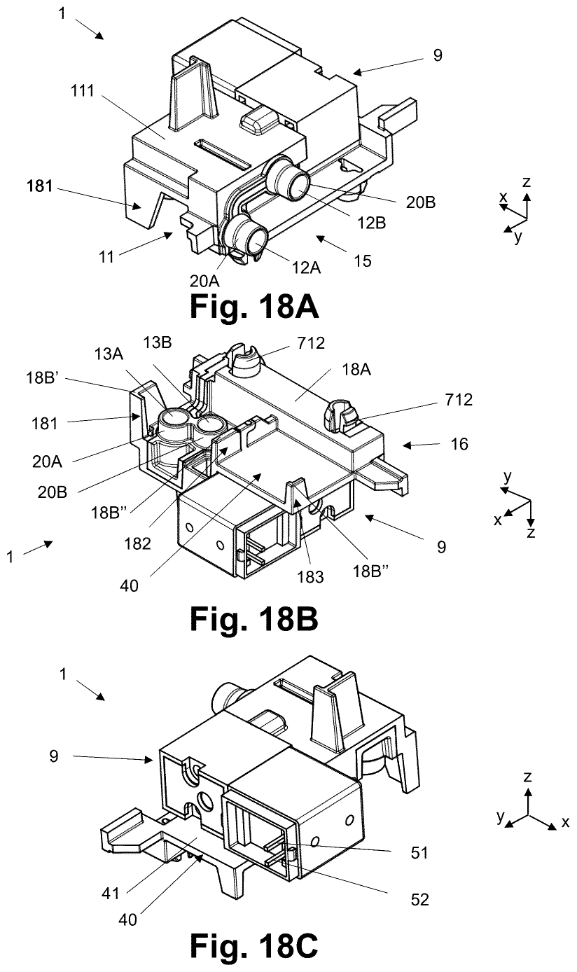

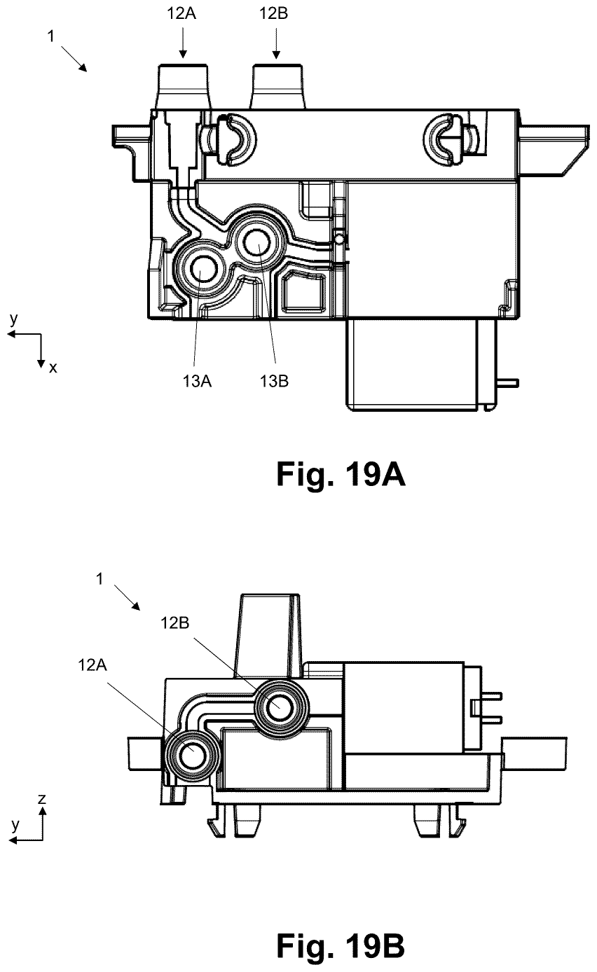

FIGS. 18 to 19 show various views of a pneumatic adapter 1 formed by using two-component molding. The pneumatic adapter 1 is configured for a single sensor (not shown), in particular a differential pressure sensor configured to be able to perform a calibration, in particular a zero-offset. The pneumatic adapter 1 is further shown connected to a three-way control valve 9, the control valve 9 configured to be controlled such one of the inlet openings 12A, 12B is fluidically connected to both outlet openings 13A, 13B, thereby ensuring that both outlet openings 13A, 13B, and therefore both pressure transducers of the sensor, are subject to the same pressure and a zero offset calibration may be performed.

For the sake of brevity, not all features (optional or not) of the pneumatic adapter 1 shown in FIG. 18 to 19 are explained. Rather, those features unique to the embodiment shown in FIG. 18 to 19 are explained. The person skilled in the art is aware that features from other embodiments in the present disclosure may be combined with the embodiment described with reference to FIG. 14 and 15 to 17.

FIG. 18A shows a first perspective view of the pneumatic adapter 1 configured for a single sensor. FIGS. 18B and 18C shows the pneumatic adapter 1 of FIG. 18A from further perspective views. The adapter body 11 comprises pneumatic sockets 20A, 20B, 20A′, 20B′, 21A, 21B, 21A′, 21B′ and an interface member 111. The properties of the adapter body 11, in particular the pneumatic sockets 20A, 20B, 20A′, 20B′, 21A, 21B, 21A′, 21B′ and the interface member 111 may be as described above with reference to FIG. 15 to 17.

The pneumatic adapter 1 is configured for a single sensor. The adapter body 11 has two inlet openings 12A, 12B on a first side 15 and two outlet openings 13A, 13B on a second side 16. The inlet openings 12A, 12B are formed by the pneumatic sockets 20A, 20B. The inlet openings 12A, 12B extend at least partially in a main direction x forming channels 14A, 14B (not seen). The outlet openings 13A, 13B of the pneumatic adapter 1 are formed by the pneumatic sockets 21A, 21B connected to the channels 14A, 14B, respectively. The pneumatic adapter 1 defines a plurality of channels 14A, 14B which extend between the inlet openings 12A, 12B, and the outlet openings 13A, 13B, thereby fluidically connecting the inlet openings 12A, 12B and outlet openings 13A, 13B. The adapter body 11, in particular the interface member 111, forms part of the channels 14A, 14B. The pneumatic sockets 21A, 21B are connected to each other by one or more bridges, which are a narrow connecting parts which extend between the pneumatic sockets 20A, 20B which form the inlet openings 12A, 12B, and between the pneumatic sockets 21A, 21B which form the outlet openings 13A, 13B, respectively at least partially along the length of the pneumatic sockets 20A, 20B, 21A, 21B. Thereby, the adapter body 11 may be formed easily using injection molding.

The pneumatic sockets 20A, 20B, 21A, 21B extend out of the adapter body 11, thereby enabling a fluidically tight connection between the pneumatic adapter 1 and the pneumatic ports and/or the sensor (not shown). The pneumatic sockets 20A, 20B, 21A, 21B may be at least partially pairwise structurally interconnected, thereby improving the manufacturability of the outlet plugs.

The interface member 111 may have a stepped support 40 which extends outwardly from a side wall in y-direction defining a support surface 41 on the opposite side of the second side 16. The interface member 111 may implement a bar which stretches below (-z) the inlet opening and the stepped support 40. The bar defines a bar surface 18A on the second side 16. The interface member 111 includes two engagement means 712 which protrude from the bar surface 18A for attaching the interface member 111 to a circuit board. In particular, each of the engagement means 712 may be implemented as a snap fit mechanism designed to engage with an opening of a circuit board, such that when the pneumatic adapter 1 is pressed onto the circuit board in the negative z-direction, the snap fit mechanisms engage and securely attach the pneumatic adapter 1 to the circuit board. While pressing the pneumatic adapter 1 onto the circuit board, the two pneumatic connectors of the sensor unit are connected to each outlet opening 13A, 13B simultaneously.

The pneumatic adapter 1 also features a first, a second and a third leg 181, 182, 183 in the vicinity of the outlet openings 13A, 13B without obstructing the outlet openings. The first, second and third leg 181, 182, 183 may protrude from the interface member 111 in the negative z-direction. In particular, the first leg 181 is located at one end of the interface member 111 in y-direction closer to the outlet openings 13A, 13B, the second leg 182 is located at the side wall from which the stepped support 40 stretches out to, and the third leg 183 protrudes at the other end of the interface member 111, in particular the stepped support 40. Each leg may include a leg surface 18B′, 18B″, 18B″ on the second side 16 which are on the same height as the bar surface 18A′, 18A. Once the engagement means 712 are connected to the circuit board, the bar surface 18A and the leg surfaces 18B′, 18B″, 18B″ at least partially come into contact with the circuit board (not shown).

The three way control valve 9 is connected to the pneumatic adapter 1. In particular, the three way control valve 9 is located at the side wall from which the stepped support 40 stretches out to. The three way control valve 9 may have two electrical pins 51, 52 for controlling the valve 9, enabling a zero-offset calibration. The pneumatic adapter 1 includes a first valve opening. The first valve opening is connected to the first inlet opening 12A by way of a first channel. The pneumatic adapter 1 includes a second valve opening. The second valve opening is connected to the second inlet opening 12B by way of a second channel (not shown). The second channel is further connected to the second outlet opening 13B (not shown). The pneumatic adapter 1 includes a third valve opening. The third valve opening is connected to a third channel which is connected to the first outlet opening 13A (not shown).

FIGS. 19A, 19B show the pneumatic adapter 1 on the second side 16 and on the first side 15 which equal a description of a sub-adapter based on FIGS. 15 to 17.

The above-described embodiments of the disclosure are exemplary and the person skilled in the art knows that at least some of the components and/or steps described in the embodiments above may be rearranged, omitted, or introduced into other embodiments without deviating from the scope of the present disclosure.

1. A pneumatic adapter for a Heating, Ventilation, and Air Condition (HVAC) sensor device, the pneumatic adapter comprising:
an adapter body made of an elastomer, the adapter body having:
two inlet openings extending into the adapter body in a main extension direction and configured to connect to two pneumatic ports of the HVAC sensor device, respectively,
two outlet openings extending into the adapter body and configured to connect to two pneumatic connectors of a sensor unit of the HVAC sensor device, the sensor unit configured for measuring at least one characteristic of an HVAC fluid; and
two channels connecting the two inlet openings to the two outlet openings respectively.

2. The pneumatic adapter of claim 1, wherein the adapter body comprises:
two pneumatic sockets defining the two inlet openings and made of the elastomer:
two pneumatic sockets defining the two outlet openings and made of the elastomer; and
an interface member made of a material different than the elastomer, the interface member forming the two channels.

3. The pneumatic adapter of claim 1, wherein the inlet openings are arranged on a first side of the adapter body, the outlet openings are arranged on a second side of the adapter body, wherein the second side is arranged substantially perpendicular to the first side.

4. The pneumatic adapter of claim 1, wherein the inlet openings are arranged on a first side of the adapter body, the outlet openings are arranged on a second side of the adapter body, wherein the second side is arranged substantially parallel to the first side.

5. The pneumatic adapter of claim 1, wherein the adapter body includes a bore extending through the adapter body in a direction perpendicular to the main extension direction of the inlet openings.

6. The pneumatic adapter of claim 1, further comprising a rigid frame arranged on at least two lateral sides of the adapter body with respect to the main extension direction.

7. The pneumatic adapter of claim 1, wherein the adapter body has a stepped contacting surface.

8. The pneumatic adapter of claim 7, wherein the stepped contacting surface has a first section which includes the bore and a second section which includes the two outlet openings.

9. The pneumatic adapter of claim 1, wherein the two inlet openings extend, in the main extension direction entirely through the adapter body forming two exit holes which are sealed by way of sealing plugs, respectively.

10. A pneumatic adapter for a Heating, Ventilation, and Air Condition (HVAC) sensor device the pneumatic adapter comprising:
an adapter body made of an elastomer, the adapter body having:
two inlet openings extending into the adapter body in a main extension direction and configured to connect to two pneumatic ports of the HVAC sensor device respectively,
two outlet openings extending into the adapter body and configured to connect to two pneumatic connectors of a sensor unit of the HVAC sensor device, the sensor unit configured for measuring at least one characteristic of an HVAC fluid;
a channel connecting a first inlet opening to a first outlet opening; and
three valve openings extending into the adapter body and configured to connect to three pneumatic connectors of a three-way control valve of the HVAC sensor device,
wherein:
a first valve opening is connected to the second inlet opening a second valve opening is connected to the second outlet opening and a third valve opening is connected to the channel.

11. A Heating, Ventilation, and Air Condition (HVAC) sensor device comprising a sensor unit configured to measure at least one characteristic of an HVAC fluid,
wherein the sensor unit is connected to the pneumatic adapter according to one claim 1.

12. A Heating, Ventilation, and Air Condition (HVAC) sensor device comprising a sensor unit configured to measure at least one characteristic of an HVAC fluid and a three-way control valve,
wherein the sensor unit and the three-way control valve are connected to the pneumatic adapter according to claim 10.

13. The HVAC sensor device of claim 11, wherein the sensor unit is configured to measure at least one of the following characteristics of an HVAC fluid: an absolute pressure, a differential pressure, a temperature, a CO2 concentration, or a particulate matter concentration.
